(19) United States
(12) Patent Application Publication
(10) Pub. No.: US 2007/0213519 A1
(43) Pub. Date: Sep. 13, 2007

(54) Building Block Forming A C=C Double Bond Upon Reaction

(75) Inventors: Alex Haahr Gouliaev, Veksoe Sjaelland (DK); Henrik Pedersen, Bagsvaerd (DK); Michael Anders Godskesen, Vedbaek (DK); Tine Titilola Akinleminu Kronborg, Copenhagen (DK); Gitte Nystrup Husemoen, Copenhagen N (DK); Justin Ho, Copenhagen V (DK); Jakob Felding, Charlottenlund (DK); Christian Sams, Frederiksberg C (DK)

Correspondence Address: BROWDY AND NEIMARK, P.L.L.C.;624 NINTH STREET, NW, SUITE 300, WASHINGTON, DC, 20001-5303, US

(73) Assignee: NUEVOLUTION A/S, VEKSOE SJAELLAND (DK)

(21) Appl. No.: 10/507,600

(22) PCT Filed: Mar. 14, 2003
(86) PCT No.: PCT/DK03/00173

§ 371 Date: May 24, 2005

Related U.S. Application Data
(60) Provisional application No. 60/361,056, filed on Mar. 1, 2002.
(60) Provisional application No. 60/434,429, filed on Dec. 19, 2002.

(30) Foreign Application Priority Data
Mar. 15, 2002 (DK) PA 2002 0415
Dec. 19, 2002 (DK) PA 2002 01952

(51) Int. Cl. C07H 21/00

(52) U.S. Cl. 536/23.1; 536/22.1

(57) ABSTRACT

A building block having the dual capabilities of transferring genetic information and functional entity precursor to a recipient reactive group is disclosed. The building block may be used in the generation of a single complex or libraries of different complexes, wherein the complex comprises an encoded molecule linked to an encoding element. Libraries of complexes are useful in the quest for pharmaceutically active compounds.

### TECHNICAL FIELD OF THE INVENTION

The present invention relates to a building block comprising a complementing element and a precursor for a functional entity. The building block is designed to transfer the functional entity precursor with an adjustable efficiency to a recipient reactive group upon recognition between the complementing element and an encoding element associated with the reactive group. The invention also relates to a method for transferring a functional entity precursor to recipient a reactive group.

### BACKGROUND

The transfer of a chemical entity from one mono-, di- or oligonucleotide to another has been considered in the prior art. Thus, N. M. Chung et al. (Biochim. Biophys. Acta, 1971, 228,536-543) used a poly(U) template to catalyse the transfer of an acetyl group from 3′-O-acetyladenosine to the 5′-OH of adenosine. The reverse transfer, i.e. the transfer of the acetyl group from a 5′-O-acetyladenosine to a 3′-OH group of another adenosine, was also demonstrated.

Walder et al. Proc. Natl. Acad. Sci. USA, 1979, 76, 51-55 suggest a synthetic procedure for peptide synthesis. The synthesis involves the transfer of nascent immobilized polypeptide attached to an oligonucleotide strand to a precursor amino acid attached to an oligonucleotide. The transfer comprises the chemical attack of the amino group of the amino acid precursor on the substitution labile peptidyl ester, which in turn results in an acyl transfer. It is suggested to attach the amino acid pre-cursor to the 5′ end of an oligonucleotide with a thiol ester linkage.

The transfer of a peptide from one oligonucleotide to another using a template is disclosed in Bruick R K et al. Chemistry & Biology, 1996, 3:49-56. The carboxy terminal of the peptide is initially converted to a thioester group and subsequently transformed to an activated thioester upon incubation with Ellman's reagent. The activated thioester is reacted with a first oligo, which is 5′-thiol-terminated, resulting in the formation of a thio-ester linked intermediate. The first oligonucleotide and a second oligonucleotide having a 3′ amino group is aligned on a template such that the thioester group and the amino group are positioned in close proximity and a transfer is effected resulting in a coupling of the peptide to the second oligonucleotide through an amide bond.

### SUMMARY OF THE INVENTION

The present invention relates to a building block of the general formula:

Complementing Element-Linker-Carrier-C-F-connecting group-Functional entity precursor

capable of transferring a Functional entity precursor to a recipient reactive group, wherein

Complementing Element is a group identifying the Functional entity precursor,
Linker is a chemical moiety comprising a spacer and a S-C-connecting group, wherein the spacer is a valence bond or a group distancing the Functional entity precursor to be transferred from the complementing element and the S-C-connecting group connects the spacer with the Carrier
Carrier is arylene, heteroarylene, C₁-C₆ alkylene, C₁-C₈ alkenylene, C₁-C₈ alkynylene, or —(CF₂)ₘ— substituted with 0-3 R¹ wherein m is an integer between 1 and 10;
   R¹ are independently selected from —H, —OR², —NR²₂, -Halogen, —NO₂, —CN, —C(Halogen)₃, —C(O)R², —C(O)NHR², C(O)NR²₂, —NC(O)R², —S(O)₂NHR², —S(O)₂NR²₂, —S(O)₂R², —P(O)₂—R², —P(O)—R², —S(O)—R², P(O)—OR², —S(O)—OR², —N+R²₃, wherein R² is H, C₁-C₈ alkyl, C₂-C₈ alkenyl, C₂-C₆ alkynyl, or aryl,
C-F-connecting group is chosen from the group consisting of —O₂—O—, —O—SO₂O—, —C(O)—O—, —S⁺(R³RRrr)—, —C—U—C(V)—O—, —P⁺(W)₂—O—, —P(W)—O— where U is —C(R²)₂—, —NR²— or —O—; V is ═O or ═NR² and W is —OR² or —N(R²)₂

Functional entity precursor is —C(H)(R³)—R⁴ or functional entity precursor is heteroaryl or aryl optionally substituted with one or more substituents belonging to the group comprising R³ and R⁴.

Wherein R³ and R⁴ independently is H, alkyl, alkenyl, alkynyl, alkadienyl, cycloalkyl, cycloheteroalkyl, aryl or heteroaryl, optionally substituted with one or more substituents selected from the group consisting of SnR⁵R⁶R⁷, Sn(OR⁵)R⁶R⁷,

Sn(OR⁵)(OR⁶)R⁷, BR⁵R⁶, B(OR⁵)R⁶, B(OR⁵)(OR⁶), halogen, CN, CNO, C(halogen)₃, OR⁵, OC(═O)R⁵, OC(═O)OR⁵, OC(═O)NR⁵R⁶, SR⁵, S(═O)R⁵, S(═O)₂R⁵, S(═O)₂NR⁵R⁶, NO₂, N₃, NR⁵R⁶, N⁺R⁶R⁶R⁷, NR⁵OR⁶, NR⁵NR⁶R⁷, NR⁵C(═O)R⁶, NR⁵C(═O)OR⁶, NR⁵C(═O)NR⁶R⁷, NC, P(═O)(OR⁵)OR⁶, P⁺R⁵R⁶R⁷, C(═O)R⁵, C(═NR⁵)R⁸, C(═NOR⁵)R⁶, C(═NNR⁵R⁶), C(═O)OR⁵, C(═O)NR⁵R⁶, C(═O)NR⁵OR⁶, C(═O)NR⁵NR⁶R⁷, C(═NR⁵)NR⁶R⁷, C(═NOR⁵)NR⁶R⁷ or R⁸,

wherein,

R⁵, R⁸, and R⁷ independently is H, alkyl, alkenyl, alkynyl, alkadienyl, cycloalkyl, cycloheteroalkyl, aryl or heteroaryl, optionally substituted with one or more substituents selected from the group consisting of halogen, CN, CNO, C(halogen)₃, ═O, OR⁸, OC(═O)R⁸, OC(═O)OR⁸, OC(═O)NR⁸R⁹, SR⁸, S(═O)R⁸, S(═O)₂R⁸, S(═O)₂NR⁸R⁹, NO₂, N₃, NR⁸R⁹, N⁺R⁸R⁹R¹⁰, NR⁵OR⁶, NR⁵NR⁶R⁷, NR⁸C(═O)R⁹, NR⁸C(═O)OR⁹, NR⁸C(═O)NR⁹R¹⁰, NC, P(═O)(OR⁸)OR⁹, P⁺R⁵R⁶R⁷, C(═O)R⁸, C(═NR⁸)R⁹, C(═NOR⁸)R⁹, C(═NNR⁸R⁹), C(═O)OR⁸, C(═O)NR⁸R⁹, C(═O)NR⁸OR⁹C(═NR⁶)NR⁶R⁷, C(═NOR⁵)NR⁶R⁷ or C(═O)NR⁸NR⁹R¹⁰, wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

wherein,

R⁸, R⁹, and R¹⁰ independently is H, alkyl, alkenyl, alkynyl, alkadienyl, cycloalkyl, cycloheteroalkyl, aryl or heteroaryl and wherein R⁸ and R⁹ may together form a 3-8 membered heterocyclic ring or R⁸ and R¹⁰ may together form a 3-8 membered heterocyclic ring or R⁹ and R¹⁰ may together form a 3-8 membered heterocyclic ring.

In the present description and claims, the direction of connections between the various components of a building block should be read left to right. For example an S-C-connecting group —C(═O)—NH— is connected to a Spacer through the carbon atom on the left and to a Carrier through the nitrogen atom on the right hand side.
The term “C₃-C₇ cycloheteroalkyl” as used herein refers to a radical of totally saturated heterocycle like a cyclic hydrocarbon containing one or more heteroatoms selected from nitrogen, oxygen, phosphor, boron and sulphur independently in the cycle such as pyrrolidine (1-pyrrolidine; 2-pyrrolidine; 3-pyrrolidine; 4-pyrrolidine; 5-pyrrolidine); pyrazolidine (1-pyrazolidine; 2-pyrazolidine; 3-pyrazolidine; 4-pyrazolidine; 5-pyrazolidine); imidazolidine (1-imidazolidine; 2-imidazolidine; 3-imidazolidine; 4-imidazolidine; 5-imidazolidine); thiazolidine (2-thiazolidine; 3-thiazolidine; 4-thiazolidine; 5-thiazolidine); piperidine (1-piperidine; 2-piperidine; 3-piperidine; 4-piperidine; 5-piperidine; 6-piperidine); piperazine (1-piperazine; 2-piperazine; 3-piperazine; 4-piperazine; 5-piperazine; 6-piperazine); morpholine (2-morpholine; 3-morpholine; 4-morpholine; 5-morpholine; 6-morpholine); thiomorpholine (2-thiomorpholine; 3-thiomorpholine; 4-thiomorpholine; 5-thiomorpholine; 6-thiomorpholine); 1,2-oxathiolane (3-(1,2-oxathiolane); 4-(1,2-oxathiolane); 5-(1,2-oxathiolane); 1,3-dioxolane (2-(1,3-dioxolane); 4-(1,3-dioxolane); 5-(1,3-dioxolane); tetrahydropyrane; (2-tetrahydropyrane; 3-tetrahydropyrane; 4-tetrahydropyrane; 5-tetrahydropyrane; 6-tetrahydropyrane); hexahydropyridazine (1-(hexahydropyridazine); 2-(hexahydropyridazine); 3-(hexahydropyridazine); 4-(hexahydropyridazine); 5-(hexahydropyridazine); 6-(hexahydropyridazine)), [1,3,2]dioxaborolane, [1,3,6,2]dioxazaborocane
The term “aryl” as used herein includes carbocyclic aromatic ring systems of 57 carbon atoms. Aryl is also intended to include the partially hydrogenated derivatives of the carbocyclic systems as well as up to four fused aromatic- or partially hydrogenated rings, each ring comprising 5-7 carbon atoms.
The term “heteroaryl” as used herein includes heterocyclic unsaturated ring systems containing, in addition to 2-18 carbon atoms, one or more heteroatoms selected from nitrogen, oxygen and sulphur such as furyl, thienyl, pyrrolyl, heteroaryl is also intended to include the partially hydrogenated derivatives of the heterocyclic systems enumerated below.
The terms “aryl” and “heteroaryl” as used herein refers to an aryl can be optionally substituted or a heteroaryl which can be optionally substituted and includes phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6 quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]uranyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl).

The Functional Entity carries elements used to interact with host molecules and optionally reactive elements allowing further elaboration of an encoded molecule of a library. Interaction with host molecules like enzymes, receptors and polymers is typically mediated through van der waal's interactions, polar- and ionic interactions and pi-stacking effects. Substituents mediating said effects may be masked by methods known to an individual skilled in the art (Greene, T. W.; Wuts, P. G. M. Protective Groups in Organic Synthesis; 3rd ed.; John Wiley & Sons: New York, 1999.) to avoid undesired interactions or reactions during the preparation of the individual building blocks and during library synthesis. Analogously, reactive elements may be masked by suitably selected protection groups. It is appreciated by one skilled in the art that by suitable protection, a functional entity may carry a wide range of substitutents.

The Functional Entity Precursor is a masked. Functional Entity that is incorporated into an encoded molecule. After incorporation, reactive elements of the Functional Entity may be revealed by un-masking allowing further synthetic operations. Finally, elements mediating recognition of host molecules may be un-masked.

In a certain aspect of the invention, Functional entity precursor is —C(H)(R¹¹)—R¹¹, or functional entity precursor is heteroaryl or aryl substituted with 0-3 R¹¹, 0-3 R¹³ and 0-3 R¹⁵, wherein

R¹¹ and R¹¹, are independently H, or selected among the group consisting of a C₁-C₈ alkyl, C₂-C₆ alkenyl, C₂-C₆ alkynyl, C₄-C₈ alkadienyl, C₃-C₇ cycloalkyl, C₃-C₇ cyclo-heteroalkyl, aryl, and heteroaryl, said group being substituted with 0-3 R¹², 0-3 R¹³ and 0-3 R¹⁵,

or R¹¹ and R¹¹, are C₁-C₃ alkylene-NR¹²₂, C₁-C₃ alkylene-NR¹²C(O)R¹⁶, C₁-C₃ alkylene-NR¹²C(O)OR¹⁶, C₁-C₂ alkylene-O—NR¹²₂, C₁-C₂ alkylene-O—NR¹²C(O)R¹⁶, C₁-C₂ alkylene-O—NR¹²C(O)OR¹⁶ substituted with 0-3 R¹⁵,

where R¹² is H or selected independently among the group consisting of C₁-C₆ alkyl, C₂-C₆ alkenyl, C₂-C₆ alkynyl, C₃-C₇ cycloalkyl, C₃-C₇ cycloheteroalkyl, aryl, heteroaryl, said group being substituted with 0-3 R¹³ and 0-3 R¹⁵,
R¹³ is selected independently from —N₃, —CNO, —C(NOH)NH₂, —NHOH, —NHNHR¹⁷, —C(O)R¹⁷, —SnR¹⁷₃, —B(OR¹⁷)₂, —P(O)(OR¹⁷)₂ or the group consisting of C₂-C₆ alkenyl, C₂-C₆ alkynyl, C₄-C₈ alkadienyl said group being substituted with 0-2 R¹⁴
where R¹⁴ is independently selected from —NO₂, —C(O)OR¹⁷, —COR¹⁷, CN, —OSiR¹⁷₃, —OR¹⁷ and —NR¹⁷₂;
R¹⁵ is ═O, —F, —Cl, —Br, —I, —CN, —NO₂, —OR¹⁷, —NR¹⁷₂, —NR¹⁷—C(O)R¹⁶, —NR¹⁷—C(O)OR¹⁶, —SR¹⁷, —S(O)R¹⁷, —S(O)₂R¹⁷, —COOR¹⁷, —C(O)NR¹⁷₂ and —S(O)₂NR¹⁷₂,
R¹⁶ is H, C₁-C₆ alkyl, C₂-C₆ alkenyl, C₂-C₆ alkynyl, C₃-C₇ cycloalkyl, aryl or C₁-C₈ alkylene-aryl substituted with 0-3 substituents independently selected from —F, —Cl, —NO₂, —R², —OR², —SiR²₃;
   R¹⁷ is selected independently from H, C₁-C₆ alkyl, C₃-C₇ cycloalkyl, aryl, C₁-C₆ alkylene-aryl, 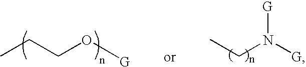
   G is H or C₁-C₈ alkyl and n is 1, 2, 3 or 4.

The function of the carrier is to ensure the transferability of the functional entity pre-cursor. To adjust the transferability a skilled chemist can design suitable substitutions of the carrier by evaluation of initial attempts. The transferability may be adjusted in response to the chemical composition of the functional entity precursor, to the nature of the complementing element, to the conditions under which the transfer and recognition is performed, etc.

In a preferred embodiment, the carrier is selected from the group consisting of arylene, heteroarylene or —(CF₂)ₘ. substituted with 0-3 R¹ wherein m is an integer between 1 and 10, and C-F-connecting group is —SO₂—O—. Due to the high reactivity of such compounds a broad range of recipient reactive groups may be employed in the construction of carbon-carbon bonds or carbon-hetero atom bonds.

In another preferred embodiment of the invention, the carrier is —(CF₂)ₘ— wherein m is an integer between 1 and 10, the C-F-connecting group is —SO₂—O—; and the functional entity precursor is aryl or heteroaryl substituted with 0-3 R¹¹, 0-3 R¹³ and 0-3 R¹⁵.

The C-F-connecting group determines in concert with the carrier the transferability of the functional entity precursor. In a preferred embodiment, the C-F-connecting group is —S⁺(R¹¹)—,

In another preferred embodiment, the C-F-connecting group is chosen from the group consisting of —SO₂O—, and —S⁺(R¹⁷)—; wherein R¹⁷ is selected independently from H, C₁-C₆ alkyl, C₃-C₇ cycloalkyl, aryl, C₁-C₆ alkylene-aryl.

In the presence of a catalyst comprising transition metals such as Pd, Ni or Cu, an aromatic moiety may be transferred from the C-F-connecting group to a recipient reactive group. Further, the transfer may be initiated by adding the catalyst, independently of the annealing of encoding—and complementing elements.

The S-C-connecting group provide a means for connecting the Spacer and the Carrier. As such it is primarily of synthetic convenience and does not influence the function of a building block.

The spacer serves to distance the functional entity precursor to be transferred from the bulky complementing element. Thus, when present, the identity of the spacer is not crucial for the function of the building block. It may be desired to have a spacer which can be cleaved by light. In this case, the spacer is provided with e.g. the group

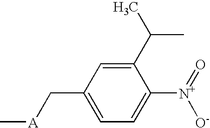

In the event an increased hydrophilicity is desired the spacer may be provided with a polyethylene glycol part of the general formula:

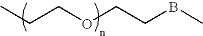

In a preferred embodiment, the complementing element serves the function of transferring genetic information e.g. by recognising a coding element. The recognition implies that the two parts are capable of interacting in order to assemble a complementing element—coding element complex. In the biotechnological field a variety of interacting molecular parts are known which can be used according to the invention. Examples include, but are not restricted to protein-protein interactions, protein-polysaccharide interactions, RNA-protein interactions, DNA-DNA interactions, DNA-RNA interactions, RNA-RNA interactions, biotin-streptavidin interactions, enzyme-ligand interactions, antibody-ligand interaction, protein-ligand interaction, etc.

The interaction between the complementing element and coding element may result in a strong or a weak bonding. If a covalent bond is formed between the parties of the affinity pair the binding between the parts can be regarded as strong, whereas the establishment of hydrogen bondings, interactions between hydrophobic domains, and metal chelation in general results in weaker bonding. In general relatively weak bonding is preferred. In a preferred aspect of the invention, the complementing element is capable of reversible interacting with the coding element so as to provide for an attachment or detachment of the parts in accordance with the changing conditions of the media.

In a preferred aspect of the invention, the interaction is based on nucleotides, i.e. the complementing element is a nucleic acid. Preferably, the complementing element is a sequence of nucleotides and the coding element is a sequence of nucleo-tides capable of hybridising to the complementing element. The sequence of nucleo-tides carries a series of nucleobases on a backbone. The nucleobases may be any chemical entity able to be specifically recognized by a complementing entity. The nucleobases are usually selected from the natural nucleobases (adenine, guanine, uracil, thymine, and cytosine) but also the other nucleobases obeying the Watson-Crick hydrogen-bonding rules may be used, such as the synthetic nucleobases disclosed in U.S. Pat. No. 6,037,120. Examples of natural and non-natural nucleobases able to perform a specific pairing are shown in FIG. 2. The backbone of the sequence of nucleotides may be any backbone able to aggregate the nucleobases is a sequence. Examples of backbones are shown in FIG. 4. In some aspects of the invention the addition of non-specific nucleobases to the complementing element is advantegeous, FIG. 3

The coding element can be an oligonucleotide having nucleobases which complements and is specifically recognised by the complementing element, i.e. in the event the complementing element contains cytosine, the coding element part contains guanine and visa versa, and in the event the complementing element contains thymine or uracil the coding element contains adenine.

The complementing element may be a single nucleobase. In the generation of a library, this will allow for the incorporation of four different functional entities into the template-directed molecule. However, to obtain a higher diversity a complementing element preferably comprises at least two and more preferred at least three nucleotides. Theoretically, this will provide for 42 and 43, respectively, different functional entities uniquely identified by the complementing element. The complementing element will usually not comprise more than 100 nucleotides. It is preferred to have complementing elements with a sequence of 3 to 30 nucleotides.

The building blocks of the present invention can be used in a method for transferring a functional entity precursor to a recipient reactive group, said method comprising the steps of
providing one or more building blocks as described above and
contacting the one or more building blocks with a corresponding encoding element associated with a recipient reactive group under conditions which allow for a recognition between the one or more complementing elements and the encoding elements, said contacting being performed prior to, simultaneously with, or subsequent to a transfer of the functional entity precursor to the recipient reactive group.

The encoding element may comprise one, two, three or more codons, i.e. sequences that may be specifically recognised by a complementing element. Each of the codons may be separated by a suitable spacer group. Preferably, all or at least a majority of the codons of the template are arranged in sequence and each of the codons are separated from a neighbouring codon by a spacer group. Generally, it is preferred to have more than two codons on the template to allow for the synthesis of more complex encoded molecules. In a preferred aspect of the invention the number of codons of the encoding element is 2 to 100. Still more preferred are encoding elements comprising 3 to 10 codons. In another aspect, a codon comprises 1 to 50 nucleotides and the complementing element comprises a sequence of nucleotides complementary to one or more of the encoding sequences.

The recipient reactive group may be associated with the encoding element in any appropriate way. Thus, the reactive group may be associated covalently or non-covalently to the encoding element. In one embodiment the recipient reactive group is linked covalently to the encoding element through a suitable linker which may be separately cleavable to release the reaction product. In another embodiment, the reactive group is coupled to a complementing element, which is capable of recognising a sequence of nucleotides on the encoding element, whereby the recipient reactive group becomes attached to the encoding element by hybridisation. Also, the recipient reactive group may be part of a chemical scaffold, i.e. a chemical entity having one or more reactive groups available for receiving a functional entity precursor from a building block.

The recipient reactive group may be any group able to participate in cleaving the bond between the carrier and the functional entity precursor to release the functional entity precursor. Typically, the recipient reactive group is a nucleophilic atom such as S, N, O, C or P. Scheme 1a shows the transfer of an alkyl group and scheme 1b shows the transfer of an vinyl group.

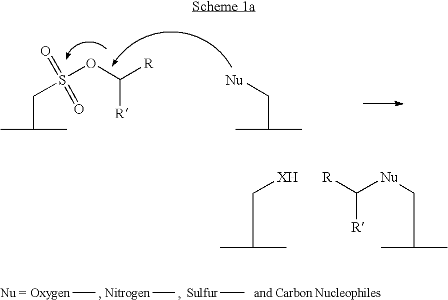

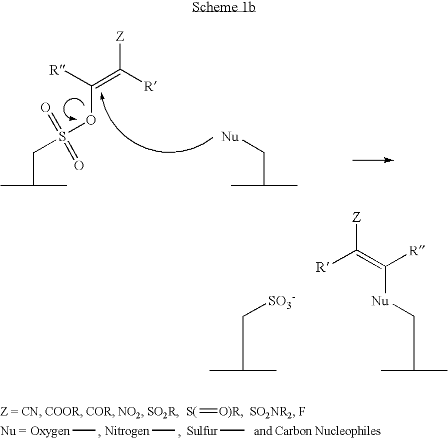

Alternatively, the recipient reactive group is a organometallic compound as shown in scheme 2.

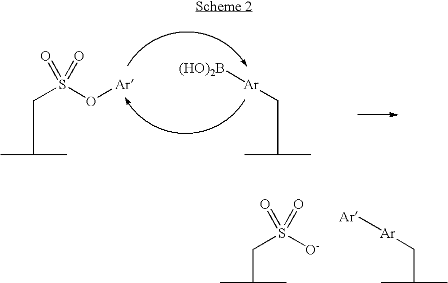

According to a preferred aspect of the invention the building blocks are used for the formation of a library of compounds. The complementing element of the building block is used to identify the functional entity. Due to the enhanced proximity between reactive groups when the complementing entity and the encoding element are contacted, the functional entity precursor together with the identity programmed in the complementing element is transferred to the encoding element associated with recipient reactive group. Thus, it is preferred that the sequence of the complementing element is unique in the sense that the same sequence is not used for another functional entity. The unique identification of the functional entity enable the possibility of decoding the encoding element in order to determine the synthetic history of the molecule formed. In the event two or more functional entities have been transferred to a scaffold, not only the identity of the transferred functional entities can be determined. Also the sequence of reaction and the type of reaction involved can be determined by decoding the encoding element. Thus, according to a preferred embodiment of the invention, each different member of a library comprises a complementing element having a unique sequence of nucleotides, which identifies the functional entity.

### BRIEF DESCRIPTION OF THE DRAWINGS

FIG. 1. Two setups for Functional Entity Transfer

FIG. 2. Examples of specific base pairing

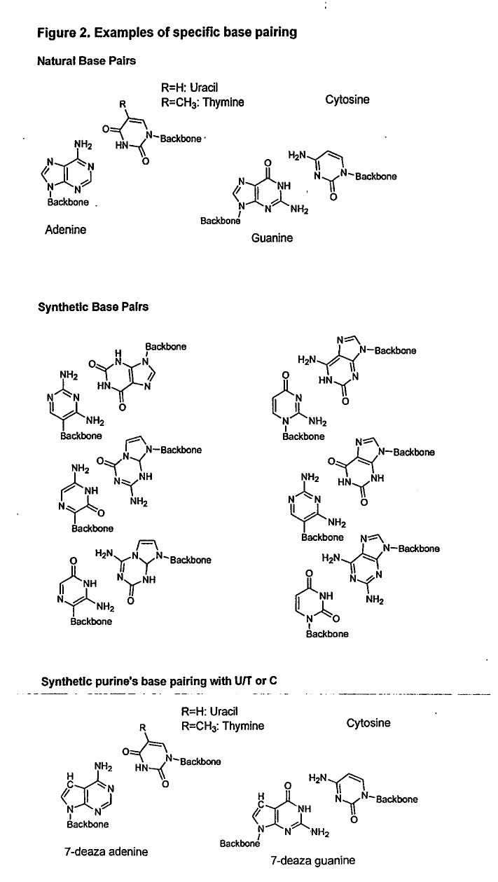

FIG. 3. Example of non-specific base-pairing

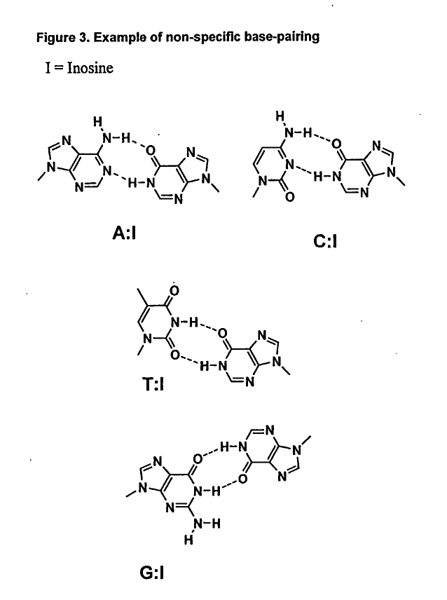

FIG. 4. Backbone examples

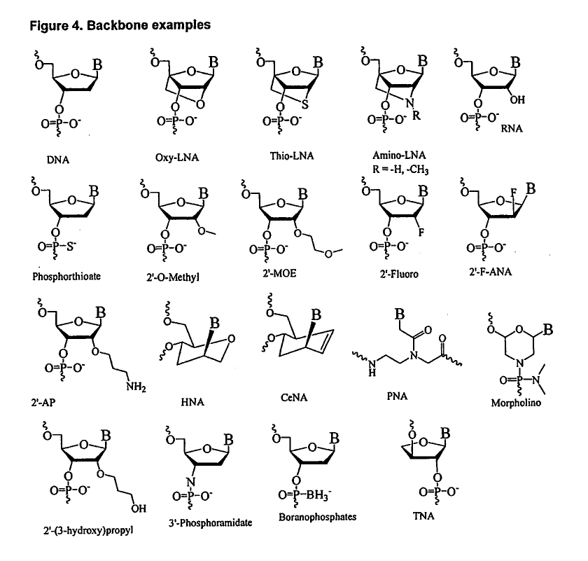

FIG. 5 Three examples of building blocks

### DETAILED DESCRIPTION OF THE INVENTION

A building block of the present invention is characterized by its ability to transfer its functional entity precursor to a recipient reactive group. This is done by forming a new covalent bond between the recipient reactive group and cleaving the bond between the carrier moiety and the functional entity precursor of the building block.

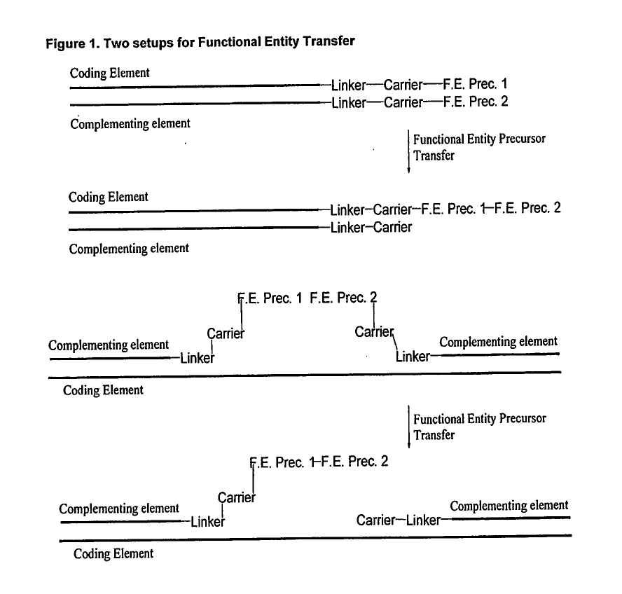

Two setups for generalized functional entity precursor transfer from a building block are depicted in FIG. 1. In the first example, one complementing element of a building block recognizes a coding element carrying another functional entity precursor, hence bringing the functional entities in close proximity. This results in a reaction between functional entity precursor 1 and 2 forming a covalent bond between these concurrent with the cleavage of the bond between functional entity precursor 2 and its linker. In the second example, a template brings together two building blocks resulting in functional entity precursor transfer from one building block to the other.

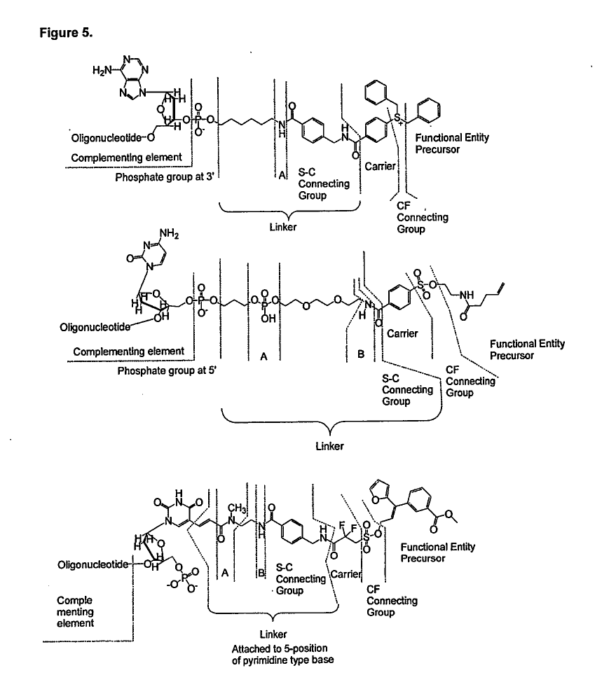

FIG. 5 illustrates three specific compounds according to the invention. For illustrative purposes the individual features used in the claims are indicated. The upper compound is an example of a building block wherein the linker is backbone attached at the 3′-position. The first part of the linker, i.e. the spacer, is an aliphatic chain ending in a nitrogen atom. The nitrogen atom bridges to the S-C-connecting group, which is an N-acylated arylmethyleamine. The carrier attached to the left hand side carbonyl group of the S-C-connecting group is a benzene ring holding the C-F Connecting group in the para position. The C-F Connecting group is a positively charged sulfur atom which is attached to the Functional Entity Precursor, in this case a benzyl group. When the building block is presented to a nucleophilic recipient reactive group, such an amine or a thiol, Functional Entity Precursor is transferred to benzylate the recipient reactive group.

The middle compound illustrates a 5′ attachment of a linker. The linker is linked through a phosphate group and extends into a three membered aliphatic chain. Through another phosphate group and a PEG linker the complementing element is linked via an amide bond to the Carrier. When the building block is presented to a nucleophile the Functional Entity Precursor is transferred resulting in an alkylation of the nucleophile.

The lower compound illustrates a nucleobase attachment of the linker. The linker attaches to the 5 position of a pyrimidine type nucleobase and extents through an α-β unsaturated N-methylated amide to the S-C-connecting group, which is a 4-amino methyl benzoic acid derivative. The functional entity precursor can be transferred to a nucleophilic recipient reactive group e.g. an amine or a thiol forming an allylic amine or thiol.

According to the invention, the functional entity precursor is of the formula —C(H)(R³)—R⁴ or functional entity precursor is heteroaryl or aryl optionally substituted with one or more substituents belonging to the group comprising R³ and R⁴. In a further preferred embodiment,

R³ and R⁴ independently is H, C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl or heteroaryl, optionally substituted with one or more substituents selected from the group consisting of SnR⁶R⁶,R⁷, Sn(OR⁵)R⁶R⁷, Sn(OR⁵)(OR⁸)R⁷, BR⁵R⁶, B(OR⁵)R⁶, B(OR⁵)(OR⁶), halogen, CN, CNO, C(halogen)₃, ═O, OR⁵, OC(═O)R⁵₃C(═O)OR⁵, OC(═O)NR⁵R⁶, SR⁵, S(═O)R⁵, S(═O)₂R⁶, S(═O)₂NR⁵R⁶, NO₂, N₃, NR⁵R⁶, N⁺R⁵R⁶R⁷, NR⁵OR⁶, NR⁵NR⁶R⁷, NR⁵C(═O)R⁶, NR⁵C(═O)OR⁶, NR⁵C(═O)NR⁶R⁷, NC, P(═O)(OR⁵)OR⁶, P⁺R⁵R⁶R⁷, C(═O)R⁵, C(═NR⁵)R⁶, C(═NOR⁵)R⁶, C(═NNR⁵R⁶), C(═O)OR⁵, C(═O)NR⁵R⁶, C(═O)NR⁵OR⁶, C(═O)NR⁵NR⁶R⁷, C(═NR⁵)NR⁶R⁷, C(═NOR⁵)NR⁶R⁷ or R⁸,

wherein,

R⁵, R⁶, R⁷ and R⁸ independently is H, C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in another preferred embodiment,

R³ and R⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of halogen, CN, C(halogen)₃, ═O, OR⁵, OC(═O)R⁵, OC(═O)OR⁵, OC(═O)NR⁵R⁶, SR⁵, S(═O)R⁵, S(═O)₂R⁵, S(═O)₂NR⁵R⁶, NO₂, NR⁵R⁶, NR⁶OR⁶, NR⁵NR⁶R⁷, NR⁵C(═O)R⁶, NR⁵C(═O)OR⁶, NR⁵C(═O)NR⁶R⁷, P(═O)(OR⁵)OR⁶, C(═O)R⁵, C(═NR⁵)R⁶, C(═NOR⁵)R⁶, C(═NNR⁵R⁶), C(═O)OR⁵, C(═O)NR⁵R⁶, C(═O)NR⁵OR⁶, C(═O)NR⁵NR⁶R⁷, C(═NR⁵)NR⁶R⁷, C(═NOR⁵)NR⁶R⁷ or R⁸,

wherein,

R⁵, R⁶, R⁷ and R⁶ independently is H, C₁-C₆ alkyl, C₃-C₇ cycloalkyl, C₃-C₇ cycloheteroalkyl, aryl or heteroaryl and wherein R⁵ and R⁶ may together form a 38 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of F, Cl, CN, CF₃, ═O, OR⁵, OC(═O)R⁵, OC(═O)OR⁵, OC(═O)NR⁵R⁶, SR⁵, S(═O)R⁵, S(═O)₂R⁵, S(═O)₂NR⁵R⁶, NO₂, NR⁵R⁸, NR⁵OR⁶, NR⁵NR⁶R⁷, NR⁵C(═O)R⁶, NR⁵C(═O)OR⁶, NR⁵C(═O)NR⁶R⁷, P(═O)(OR⁵)OR⁵, C(═O)R⁵, C(═NR⁵)R⁶, C(═NOR⁵)R⁶, C(═NNR⁵R⁶), C(═O)OR⁵, C(═O)NR⁵R⁶, C(═O)NR⁵OR⁶, C(═O)NR⁵NR⁶R⁷, C(═NR⁵)NR⁶R⁷, C(═NOR⁵)NR⁶R⁷ or R⁸,

wherein,

R⁵, R⁶, R⁷ and R⁵ independently is H, C₁-C₆ alkyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of F, Cl, CN, CF₃, ═O, OR⁵,S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁶, C(═O)NR⁵R⁶, C(═O)NR⁵OR⁶ or R⁸,

wherein,

R⁵, R⁶, R⁷ and R⁸ independently is H, C₁-C₆ alkyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, methyl, ethyl, propyl, butyl, cyclopropyl, cyclobutyl, cyclopentyl, cyclohexyl, aziridinyl, azetidinyl, pyrrolidinyl, piperidinyl, morpholinyl, phenyl, naphtyl, thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, C₁-C₆ alkyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another prefered embodiment,

R³ and R⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁶C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, C₁-C₆ alkyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, aziridinyl, azetidinyl, pyrrolidinyl, piperidinyl or morpholinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁶,

wherein,

R⁵, R⁶, R⁷ and R⁸ independently is H, C₁-C₆ alkyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring, in still another prefered embodiment, R³ and R⁴ independently is H, phenyl, naphtyl, thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR^{o}C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, C₁-C₆ alkyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁵ and R⁷ may together form a 3-8 membered heterocyclic ring,

in still another prefered embodiment,

R³ and R⁴ independently is H, phenyl or naphtyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵⁰, R⁶ or R⁸,

wherein,

R⁵, R⁶, R⁷ and R⁸ independently is H, C₁-C₆ alkyl, C₃-C₇ cycloalkyl, C₃-C₇ cycloheteroalkyl, aryl or heteroaryl and wherein R⁵ and R³ may together form a 3-8 membered heterocyclic ring or R⁵ and R⁷ may together form a 3-8 membered heterocyclic ring or R⁶ and R⁷ may together form a 3-8 membered heterocyclic ring, in still another prefered embodiment, R³ and R⁴ independently is H, thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R^{e}, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁶, R⁶, R⁷ and R⁸ independently is H, C₁-C₆ alkyl, C₃-C₇ cycloalkyl, C₃-C₇ cycloheteroalkyl, aryl or heteroaryl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another prefered embodiment,

R³ and R⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, aziridinyl, azetidinyl, pyrrolidinyl, piperidinyl or morpholinyl optionally substituted with one or more substituents selected from the group consisting of F, Cl, CN, CF₃, ═O, ORG, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, phenyl, naphtyl, thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁸, R⁷ and R⁸ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, phenyl or naphtyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R⁵ and R⁶ may together form a 3-8 membered heterocyclic ring or R⁵ and R⁷ may together form a 3-8 membered heterocyclic ring or R⁵ and R⁷ may together form a 3-8 membered heterocyclic ring,

in still another preferred embodiment,

R³ and R⁴ independently is H, thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵R⁶ or R⁸,

wherein,

R⁵, R⁶, R⁷ and R⁸ independently is H, methyl, ethyl, propyl or butyl and wherein R⁵ and R⁶ may together form a 3-8 membered heterocyclic ring or R⁵ and R⁷ may together form a 3-8 membered heterocyclic ring or R⁵ and R⁷ may together form a 3-8 membered heterocyclic ring,

in still another preferred embodiment,

R³ and R⁴ independently is H, aziridinyl, azetidinyl, pyrrolidinyl, piperidinyl or morpholinyl optionally substituted with one or more substituents selected from the group consisting of F, Cl, CN, CF₃, ═O, OR⁶, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁵R⁶, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, methyl, ethyl, propyl or butyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, phenyl, naphtyl, thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁶C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, methyl, ethyl, propyl or butyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, phenyl or naphtyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵R⁶ or R⁸,

wherein,

R⁵, R⁶, R⁷ and R⁸ independently is H, methyl, ethyl, propyl or butyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is H, thienyl, furyl, pyridyl, quinolinyl or isoquinolinyl optionally substituted with one or more substituents selected from the group consisting of F, Cl, CN, CF₃, ═O, OR⁵, S(═O)R⁶, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, methyl, ethyl, propyl or butyl and wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,

in still another preferred embodiment,

R³ and R⁴ independently is methyl, ethyl, propyl, butyl, cyclopropyl, cyclobutyl, cyclopentyl or cyclohexyl optionally substituted with one or more substituents selected from the group consisting of F, Cl, CN, CF₃, ═O, OR⁵, S(═O)R⁵, S(═O)₂R⁵, S(═O)₂NR⁵R⁶₃ NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, cyclopropyl, cyclobutyl, cyclopentyl or cyclohexyl,

in still another preferred embodiment,

R³ and R⁴ independently is aziridinyl, azetidinyl, pyrrolidinyl, piperidinyl or morpholinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵R⁶ or R⁸,

wherein,

R⁵, R⁶, R⁷ and R⁸ independently is H, cyclopropyl, cyclobutyl, cyclopentyl or cyclohexyl,

in still another preferred embodiment,

R³ and R⁴ independently is phenyl, naphtyl, thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁶)R⁶, C(═O)OR⁵, C(═O)NR⁵R⁶, C(═O)NR⁵OR⁶ or R⁸,

wherein,

R⁵, R⁸, R⁷ and R⁸ independently is H, cyclopropyl, cyclobutyl, cyclopentyl or cyclohexyl,

in still another preferred embodiment,

R³ and R⁴ independently is phenyl or naphtyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁶C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁶, R⁸, R⁷ and R⁸ independently is H, cyclopropyl, cyclobutyl, cyclopentyl or cyclohexyl,

in still another preferred embodiment,

R³ and R⁴ independently is thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁷C(═O)NR⁶R⁷, C(═O)R⁵, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, cyclopropyl, cyclobutyl, cyclopentyl or cyclohexyl,

in still another preferred embodiment,

R³ and R⁴ independently is methyl, ethyl, propyl, butyl, cyclopropyl, cyclobutyl, cyclopentyl or cyclohexyl optionally substituted with one or more substituents selected from the group consisting of F, Cl, CN, CF₃, ═O, OR⁶, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁸, C(═O)OR⁵, C(═O)NR⁵R⁶, C(═O)NR⁵OR⁶ or R⁸,

wherein,

R⁵, R⁶, R⁷ and R⁸ independently is H, phenyl, naphthyl, thienyl, furyl, pyridinyl, quinolinyl or isoquinolinyl,

in still another preferred embodiment,

R³ and R⁴ independently is aziridinyl, azetidinyl, pyrrolidinyl, piperidinyl or morpholinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁶, C(═NOR⁵)R⁶, C(═O)OR⁵, C(═O)NR⁵R⁶, C(═O)NR⁵OR⁶ or R⁸,

wherein,

R⁵, R⁶, R⁷ and R⁸ independently is H, phenyl, naphthyl, thienyl, furyl, pyridinyl, quinolinyl or isoquinolinyl,

in still another preferred embodiment,

R³ and R⁴ independently is phenyl, naphtyl, thienyl, furyl, pyridyl, quinolinyl or isoquinolinyl optionally substituted with one or more substituents selected from the group consisting of F, Cl, CN, CF₃, ═O, OR⁵, S(═O)R⁵S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁶, C(═O)NR⁵R⁶, C(═O)NR⁵OR⁶ or R⁶,

wherein,

R⁶, R⁶, R⁷ and R⁸ independently is H, phenyl, naphthyl, thienyl, furyl, pyridinyl, quinolinyl or isoquinolinyl,

in still another preferred embodiment, R³ and R⁴ independently is phenyl or naphtyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁵)R⁶, C(═O)OR⁵, C(═O)NR⁵R⁶, C(═O)NR⁵OR⁶

wherein,

R⁵, R⁶, R⁷ and R⁸ independently is H, phenyl, naphthyl, thienyl, furyl, pyridinyl, quinolinyl or isoquinolinyl,

in still another preferred embodiment,

R³ and R⁴ independently is thienyl, furyl, pyridyl, quinolinyl or isoquinolinyl optionally substituted with one or more substituents selected from the group consisting of F, Cl, CN, CF₃, ═O, OR⁵, S(═O)R⁵, S(═O)₂R⁵, S(═O)₂NR⁵R⁶, NO₂, NR⁵R⁶, NR⁵C(═O)R⁶, NR⁵C(═O)OR⁶, NR⁵C(═O)NR⁶R⁷, C(═O)R⁵, C(═NOR⁶)R⁸, C(═O)OR⁶, C(═O)NR⁵R⁶, C(═O)NR⁵OR⁶ or R⁸,

wherein,

R⁵, R⁶, R⁷ and R⁸ independently is H, phenyl, naphthyl, thienyl, furyl, pyridinyl, quinolinyl or isoquinolinyl,

in still another preferred embodiment,

R³ and R⁴ independently is H, C₁-C₆ alkyl, C₃-C₇ cycloalkyl, C₃-C₇ cycloheteroalkyl, aryl or heteroaryl

in still another preferred embodiment,

R³ and R⁴ independently is H, in still another prefered embodiment, R³ and R⁴ independently is C₁-C₆ alkyl, C₃-C₇ cycloalkyl or C₃-C₇ cycloheteroalkyl,

in still another preferred embodiment,

R³ and R⁴ independently is methyl, ethyl, propyl or butyl

in still another preferred embodiment

R³ and R⁴ independently is cyclopropyl, cyclobutyl, cyclopentyl or cyclohexyl

in still another preferred embodiment

R³ and R⁴ independently is aziridinyl, pyrrolidinyl, piperidinyl or morpholinyl

in still another preferred embodiment,

R³ and R⁴ independently is aryl or heteroaryl

in still another preferred embodiment,

R³ and R⁴ independently is phenyl or naphthyl

in still another preferred embodiment,

R³ and R⁴ independently is thienyl, furyl, pyridyl, quinolinyl or isoquinolyl

Experimental Section

General Procedure 1: Preparation of Carrier-Functional Entity Reagents:

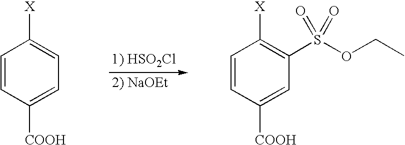

The 4-halobenzoic acid (25 mmol) is added to a ice cooled solution of chloro sulfonic acid (140 mmol). The mixture is slowly heated to reflux and left at reflux for 2-3 hours. The mixture is added to 100 mL ice and the precipitate collected by filtration. The filtrate is washed with water (2×50 mL) and the dried in vacuo affording the corresponding sulfonoyl chloride in 60-80% yield. The 3-chlorosulfonyl-4-halobenzoic acid derivate (5 mmol) is dissolved in EtOH (5 mL) and added to a ice cooled mixture of NaOEt (10 mL, 2M). The mixture is stirred o/n at rt. Acetic acid (40 mmol) is added and the mixture is evaporated in vacuo. Water (10 mL) is added and pH adjusted to pH=2 (using 1 M HCl). The product is extracted with DCM (2×25 mL), dried over Na₂SO₄ and evaporated in vacuo affording the desired products.

### EXAMPLE 1 (GENERAL PROCEDURE (1))

### 3-Ethoxysulfonyl-4-fluorobenzoic acid

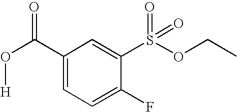

¹H-NMR (DMSO-d₆): δ 8.49 (d, 1H), 7.85 (dd, 1H), 7.5 (d, 1H), 4.32 (q. 2H), 1.32 (t, 3H)

### EXAMPLE 2 (GENERAL PROCEDURE (1))

### 4-chloro-3-Ethoxysulfonylbenzoic acid

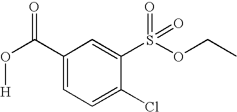

¹H-NMR (DMSO-d₆): δ 8.49 (d, 1H), 7.85 (dd, 1H), 7.5 (d, 1H), 4.32 (q, 2H), 1.32 (t, 3H)

### EXAMPLE 3

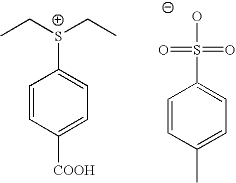

4-Methylsulfanyl benzoic acid (0.5 g, 2.97 mmol, commercially available from Aldrich, cat no. 145521) was added to methyl p-toluene solfunate (0.61 g, 3.27 mmol). The mixture was heated to 140° C. for 1 hour in a sealed vessel. After cooling to rt the mixture was trituated with diethyl ether. Filtration and drying in vacuo yielded 844 mg (80%) of the desired product (>95% pure by ¹H nmr).

¹H nmr (DMSO-d₆): 8.20-8.10 (m, 4H), 7.45 (d, 2H), 7.08 (d, 2H), 3.29 (s, 6H), 2.30 (s, 3H).
General Procedure 2: Solid Phase Preparation of Carrier-Functional Entity Reagents for Alkylation Building Blocks:

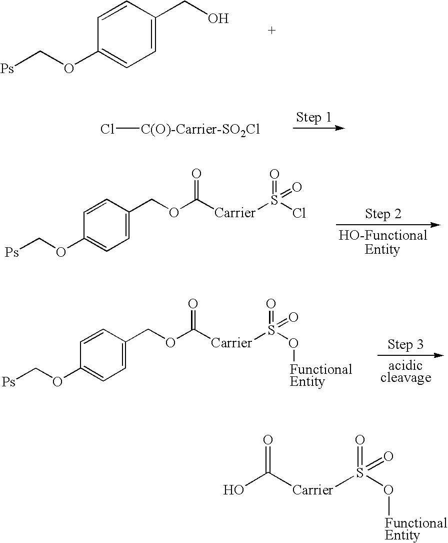

Ps=Polystyrene resin. Alternatively other acid labile linkers may be employed.

Step 1:

A polystyrene resin with a wang linker (4-hydroxymethylphenol linker) (50 mg˜50 umol), a bifunctional carrier (200 umol, 4 equiv) in a solvent such as THF, DCM, DCE, DMF, NMP or a mixture thereof (500 uL) and a base such as TEA, DIEA, pyridine (400 umol, 8 equiv), optionally in the presence of DMAP (100 umol), are allowed to react at temperatures between −20 DC and 60 DC, preferably between 0 DC and 25 DC, for 1-24 h, preferably 1-4 h. The resin is washed with the solvent composition used during the reaction (5×1 mL) and used in the following step.

Step 2:

A functional entity precursor carrying a hydroxy group in the position of the intended attachment to the C-F-connecting group (200 umol, 4 equiv) in a solvent such as THF, DCM, DCE, DMF, NMP or a mixture thereof (500 uL) and a base such as TEA, DIEA, pyridine (400 umol, 8 equiv), optionally in the presence of DMAP, are added to the resin bound carrier isolated in step 1 and allowed to react at temperatures between 0 DC and 100 DC, preferably between 25 DC and 80 DC, for 2-48 h, preferably 4-16 h. The resin is washed with the solvent composition used during the reaction (5×1 mL).

Step 3:

The desired Carrier-Functional entity reagent is cleaved from the resin obtained in step 2 by treatment with an acid like TFA, HF or HCl in a solvent such as THF, DCM, DCE or a mixture thereof (1 mL) at temperatures between −20° C. and 60° C., preferably between 0° C. and 25° C., for 1-4 h, preferably 1-2 h. Upon filtration, the resin is washed with the solvent composition used during cleavage (2×1 mL) and the combined filtrates are evaporated in vacuo. The isolated product may be purified by chromatography.

Assembly of Building Blocks

The Carrier-Functional entity reagent may be bound to the Spacer by several different reactions as illustrated below.
Formation of an Amide Bond between a Carboxylic Acid of the Carrier and an Amine Group of a Spacer 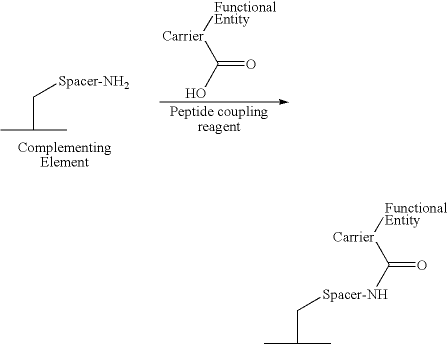
General Procedure 3: Preparation of Building Blocks by Loading a Carder-Functional Entity Reagent onto a Nucleotide Derivative Comprising an Amino Group:

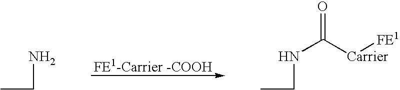

15 μL of a 150 mM building block solution of FE¹-Carrier-COOH is mixed with 15 μL of a 150 mM solution of EDC and 15 μL of a 150 mM solution of N-hydroxy-succinimide (NHS) using solvents like DMF, DMSO, water, acetonitril, THF, DCM, methanol, ethanol or a mixture thereof. The mixture is left for 15 min at 25° C. 45 μL of an aminooligo (10 nmol) in 100 mM buffer at a pH between 5 and 10, preferably 6.0-7.5 is added and the reaction mixture is left for 2 hours at 25° C. Excess building block and organic by-products were removed by extraction with EtOAc (400 μL). Remaining EtOAc is evaporated in vacuo using a speedvac. The building block is purified following elution through a BioRad micro-spin chromatography column, and analyzed by electron spray mass spectrometry (ES-MS).

### EXAMPLE 4 (GENERAL PROCEDURE ( ))

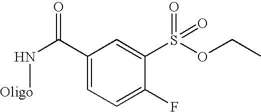 Where Oligo is 5′ XCG ATG GAT GCT CCA GGT CGC 3′, X=5′ amino C6 (Glen catalogue# 10-1906-90), Expected molecular weight: 6313.22

MS (calc.)=6543,43; MS (found)=6513,68*
* Observed molecular weight of the cleaved sulfonic ester: 6513.68 Expected molecular weight of the deaved ester: 6514.37 The quantitative loss of the ethyl group is probably due to the presence of piperidine during the recording of the LC-MS data.

General Procedure 4: Loading of a Carrier Coupled Functional Entity onto an Amino Oligo:

25 μl 100 mM carrier coupled functional entity dissolved in DMF (dimethyl form amide) was mixed with 25 μl 100 mM EDC (1-ethyl-3(3-dimethylaminopropyl) carbodiimide hydrochloride) in DMF for 30 minutes at 25° C. The mixture was added to 50 μl amino oligo in H₂O with 100 mM HEPES (2-[4-(2-hydroxy-ethyl)-piperazin-1-yl]-ethanesulfonic acid) pH 7.5 and the reaction was allowed to proceed for 20 minutes at 25° C. Unreacted carrier coupled functional entity was removed by extraction with 500 μl EtOAc (ethyl acetate), and the oligo was purified by gel filtration through a microspin column equilibrated with 100 mM MES (2-(N-morpholino) ethanesulfonic acid) pH 6.0.

Oligonucleotide Used:

Oligo A: 5′-YACGATGGATGCTCCAGGTCGC

γ=Amino modifier C6 (Glen# 10-1906)

### EXAMPLE 5 (GENERAL PROCEDURE 4)

Carrier—Functional Entity: (4-Carboxy-phenyl)-dimethyl-sulfonium

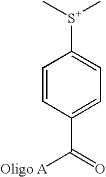

Mass: 6789.21 (observed using ES-MS), 6790.65 (calculated)
General Procedure 5: Preparation of Arylation Building Blocks: 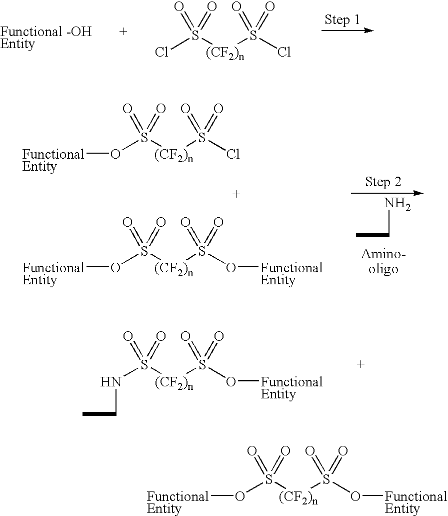
Functional Entity-OH is a phenol, n is an Integer between 3 and 6.
Step 1

To a solution of the bis-sulfonylchloride (Ward, R. B.; J. Org. Chem.; 30; 1965; 3009-3011; Qiu, Weiming; Burton, Donald J.; J. Fluorine Chem.; 60; 1; 1993; 93-100) (3 umol) in DMF, DMSO, acetonitril, THF or a mixture thereof (150 uL) is a phenolic functional entity in excess (1.05-1.8 mmol) in DMF, DMSO, acetonitril, THF or a mixture thereof (150 uL) added slowly at temperatures between −20° C. and 100° C. preferably at 0-50° C. in the presence of a base such as TEA, DIEA, pyridine, Na—HCO₃ or K₂CO₃.

Step 2

The reaction mixture from step 1 is added to a solution of an aminooligo (10 nmol) in 100 mM buffer at a pH between 5 and 10, preferably 6.0-7.5 optionally in the presence of NHS. The reaction mixture is left for 2 hours at 25° C. Excess building block and organic by-products were removed by extraction with EtOAc (400 μL). Remaining EtOAc is evaporated in vacuo using a speedvac. The building aminooligo is purified following elution through a BioRad micro-spin chromatography column, and analyzed by electron spray mass spectrometry (ES-MS).

Use of Building Blocks
General Procedure 6: Alkylation of Oligonucleotide Derivatives Containing a Nucleophilic Recipient Group using a Building Block of the Invention:

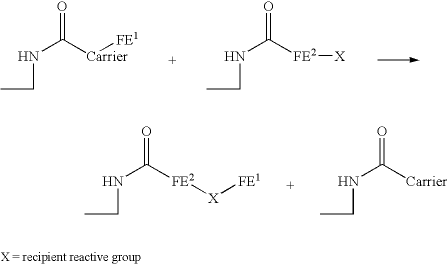

An oligonucleotide building block carrying functional entity FE¹ is combined at 2 μM final concentration with one equivalent of a complementary building block displaying a nucleophilic recipient group. Reaction proceeds at temperatures between 0° C. and 100° C. preferably between 15° C.-50° C. for 1-48 hours, preferably 10-20 hours in DMF, DMSO, water, acetonitril, THF, DCM, methanol, ethanol or a mixture thereof, pH buffered to 4-10, preferably 6-8. Organic by-products are removed by extraction with EtOAc, followed by evaporation of residual organic solvent for 10 min in vacuo. Pd catalyst is removed and oligonucleotides are isolated by eluting sample through a BioRad micro-spin chromatography column. Coupling efficiency is quantified by ES-MS analysis.

General procedure 7: Transfer of Functional Entity from a Carrier Oligo to Recipient Reactive Group.

A carrier coupled functional entity oligo (Example 1) (250 μmol) was added to a scaffold oligo B (200 μmol) in 50 μl 100 mM MES, pH 6. The mixture was incubated overnight at 25° C. Subsequently, the mixture was purified by gel filtration using a microspin column equilibrated with H₂O and transfer of the functional entity was verified by electron spray mass spectrometry (ES-MS). Transfer efficiency is expressed in percent and were calculated by dividing the abundance of scaffold oligo carrying transferred functional entities to total abundance of scaffold oligos (with and without transferred functional entities).

### EXAMPLE 6 (GENERAL PROCEDURE 7)

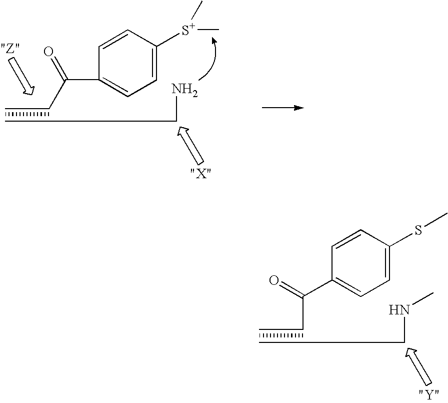

Mass (“X”): 6583.97 (observed), 6583.31 (calculated). Abundance: 65.79 (arbitrary units)

Mass (“Y”): 6599.73 (observed), 6597.34 (calculated). Abundance: 29.23 (arbitrary units)

Mass (“Z”): 6789.36 (observed), 6790.65 (calculated)

Transfer efficiency calculated as: 29.23/(29.23+65.79)=0.3076˜31%
General Procedure 8: Arylation of Oligonucleotide Derivatives Containing a Nucleophilic Recipient Group using a Building Block of the Invention:

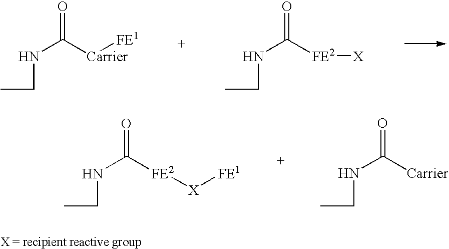

An oligonucleotide building block carrying functional entity FE¹ is combined at 2 μM final concentration with one equivalent of a complementary building block displaying a nucleophilic recipient group. In the presence of a Pd catalyst, the reaction proceeds at temperatures between 0° C. and 100° C. preferably between 15° C.-50° C. for 1-48 hours, preferably 10-20 hours in DMF, DMSO, water, acetonitrile, THF, DCM, methanol, ethanol or a mixture thereof, pH buffered to 4-10, preferably 68. Organic by-products are removed by extraction with EtOAc, followed by evaporation of residual organic solvent for 10 min in vacuo. Pd catalyst is removed and oligonucleotides are isolated by eluting sample through a BioRad micro-spin chromatography column. Coupling efficiency is quantified by ES-MS analysis.
General Procedure 9: General Route to the Formation of Alkylating/Vinylating Monomer Building Blocks with a Thio-Succinimid S-C-Connecting Group and use of these:

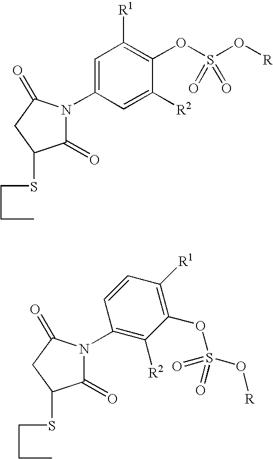

R¹=H, Me,Et, iPr, Cl, NO₂

R²=H, Me, Et, iPr, Cl, NO₂

R¹ and R² may be used to tune the reactivity of the sulphate to allow appropriate reactivity. Chloro and nitro substitution will increase reactivity. Alkyl groups will decrease reactivity. Ortho substituents to the sulphate will due to steric reasons direct incoming nucleophiles to attack the R-group selectively and avoid attack on sulphur. E.g.

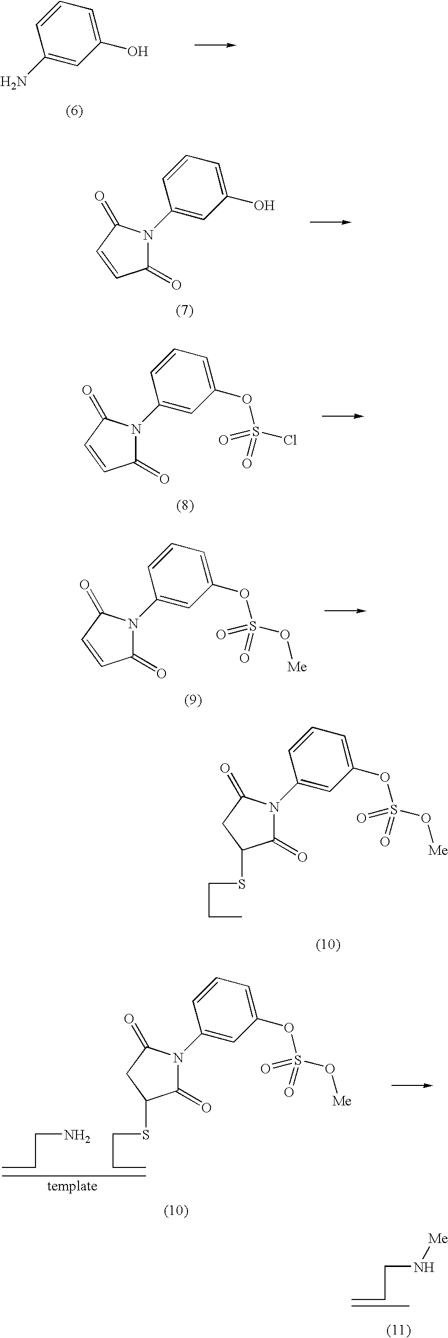

3-Aminophenol (6) is treated with maleic anhydride, followed by treatment with an acid e.g. H₂SO₄ or P₂O₅ and heat to yield the maleimide (7). The ring closure to the maleimide may also be achieved when an acid stable O-protection group is used by treatment with or Ac₂O with or without heating, followed by O-deprotection. Alternatively reflux in Ac₂O, followed by O-deacetylation in hot water/dioxane to yield (7).

Further treatment of (7) with SO₂Cl₂ with or without triethylamine or potassium carbonate in dichloromethane or a higher boiling solvent will yield the intermediate (8), which may be isolated or directly further transformed into the aryl alkyl sulphate by the quench with the appropriate alcohol, in this case MeOH, whereby (9) will be formed. The organic building block (9) may be connected to an oligo nucleotide, as follows.

A thiol carrying oligonucleotide in buffer 50 mM MOPS or hepes or phosphate pH 7.5 is treated with a 1-100 mM solution and preferably 7.5 mM solution of the organic building block (9) in DMSO or alternatively DMF, such that the DMSO/DMF concentration is 5-50%, and preferably 10%. The mixture is left for 1-16 h and preferably 24 h at 25° C. To give the alkylating in this case methylating monomer building block (10).

The reaction of the alkylating monomer building block (10) with an amine carrying monomer building block may be conducted as follows:

The coding oligonucleotide (1 nmol) is mixed with a thio oligonucleotide loaded with a building block (1 nmol) (10) and an amino-oligonucleotide (1 nmol) in hepes-buffer (20 μL of a 100 mM hepes and 1 M NaCl solution, pH=7.5) and water (39 uL). The oligonucleotides are annealed to the template by heating to 50° C. and cooled (2° C./second) to 30° C. The mixture is then left o/n at a fluctuating temperature (10° C. for 1 second then 35° C. for 1 second), to yield the template bound methylamine (11).

A vinylating monomer building block may be prepared and used similarily as described above for an alkylating monomer building block. Although instead of reacting the chlorosulphonate (8 above) with an alcohol, the intermediate chlorosulphate is isolated and treated with an enolate or O-trialkylsilylenolate with or without the presence of fluoride. E.g. 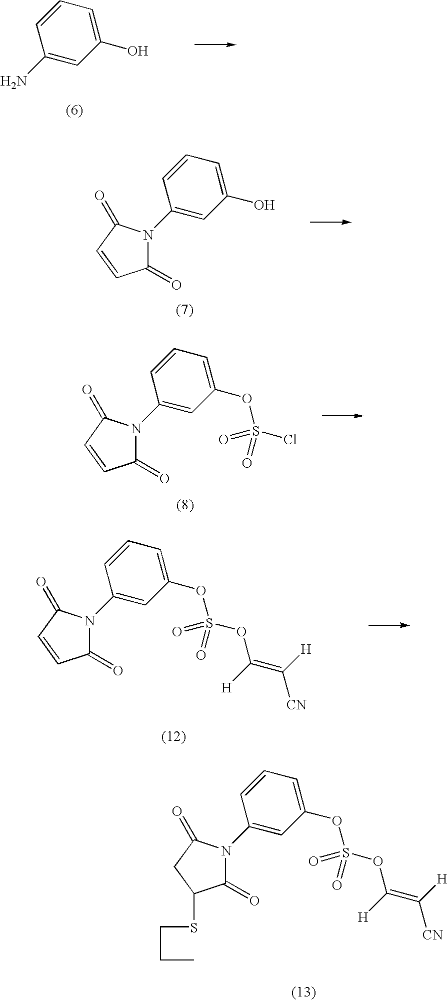
Formation of the Vinylating Monomer Building Block (13):

The thiol carrying oligonucleotide in buffer 50 mM MOPS or hepes or phosphate pH 7.5 is treated with a 1-100 mM solution and preferably 7.5 mM solution of the organic building block (12) in DMSO or alternatively DMF, such that the DMSO/DMF concentration is 5-50%, and preferably 10%. The mixture is left for 1-16 h and preferably 2-4 h at 25° C. To give the vinylating monomer building block (13).

The sulfonylenolate (13) may be used to react with amine carrying monomer building block to give an enamine (14a and/or 14b) or e.g. react with an carbanion to yield (15a and/or 15b). E.g.

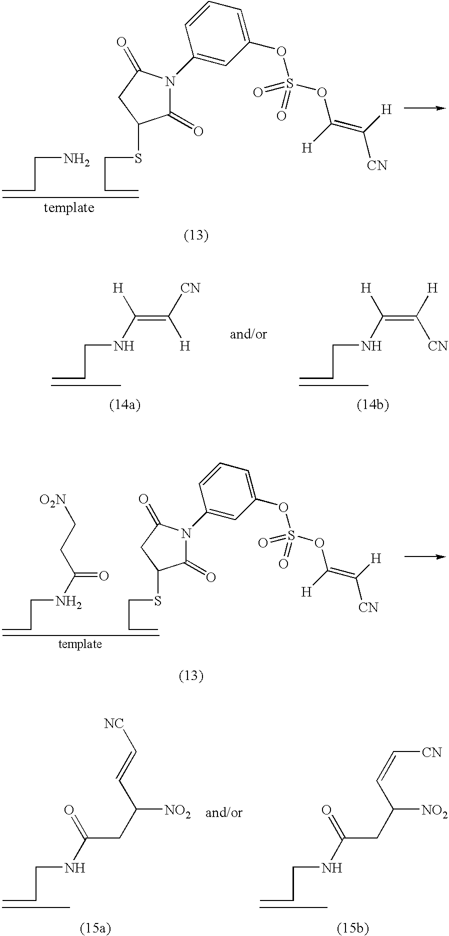

The reaction of the vinylating monomer building block (13) and an amine or nitroalkyl carrying monomer building block may be conducted as follows:

The coding oligonucleotide (1 nmol) is mixed with a oligonucleotide building block (1 nmol) (13) and an amino-oligonucleotide (1 nmol) or nitroalkyl-oligonucleotide (1 nmol) in 0.1 M TAPS, phosphate or hepes-buffer and 300 mM NaCl solution, pH=7.5-8.5 and preferably pH=8.5. The oligonucleotides are annealed to the template by heating to 50° C. and cooled (2° C./second) to 30° C. The mixture is then left o/n at a fluctuating temperature (10° C. for 1 second then 35° C. for 1 second), to yield template bound (14a/b or 15a/b).

Abbreviations

| | |
|---|---|
| DCC | N,N′-Dicyclohexylcarbodiimide |
| DhbtOH | 3,4-dihydro-3-hydroxy-4-oxo-1,2,3-benzotriazine |
| DIC | Diisopropylcarbodiimide |
| DIEA | Diethylisopropylamin |
| DMAP | 4-Dimethylaminopyridine |
| DNA | Deoxyribosenucleic Acid |
| EDC | 1-Ethyl-3-(3′-dimethylaminopropyl)carbodiimide•HCl |
| HATU | 2-(1H-7-Azabenzotriazole-1-yl)-1,1,3,3-tetramethyluronium |
| | hexafluorophosphate |
| HBTU | 2-(1H-Benzotriazole-1-yl)-1,1,3,3-tetramethyluronium |
| | hexafluorophosphate |
| HOAt | N-Hydroxy-7-azabenzotriazole |
| HOBt | N-Hydroxybenzotriazole |
| LNA | Locked Nucleic Acid |
| NHS | N-hydroxysuccinimid |
| OTf | Trifluoromethylsulfonate |
| OTs | Toluenesulfonate |
| PNA | Peptide Nucleic Acid |
| PyBoP | Benzotriazole-1-yl-oxy-tris-pyrrolidino-phosphonium |
| | hexafluorophosphate |
| PyBroP | Bromo-tris-pyrrolidino-phosphonium hexafluorophosphate |
| RNA | Ribonucleic acid |
| TBTU | 2-(1H-Benzotriazole-1-yl)-1,1,3,3-tetramethyluronium tetra- |
| | fluoroborate |
| TEA | Triethylamine |
| RP-HPLC | Reverse Phase High Performance Liquid Chromatography |
| TBDMS-Cl | Tert-Butyldimethylsilylchloride |
| 5-Iodo-dU | 5-iodo-deoxyriboseuracil |
| TLC | Thin layer chromatography |
| (Boc)₂O | Boc anhydride, di-tert-butyl dicarbonate |
| TBAF | Tetrabutylammonium fluoride |
| SPDP | Succinimidyl-propyl-2-dithiopyridyl |

1. A building block of the general formula
Complementing Element-Linker-Carrier-C-F-connecting group-Functional entity precursor
capable of transferring a Functional entity precursor to a recipient reactive group, wherein
Complementing Element is a group identifying the Functional entity precursor,
Linker is a chemical moiety comprising a spacer and a S-C-connecting group, wherein the spacer is a valence bond or a group distancing the Functional entity precursor to be transferred from the complementing element and the S-C-connecting group connects the spacer with the Carrier
Carrier is arylene, heteroarylene, C₁-C₆ alkylene, C₁-C₆ alkenylene, C₁-C₆ alkynylene, or —(CF₂)ₘ substituted with 0-3 R¹ wherein m is an integer between 1 and 10;
R¹ are independently selected from —H, —OR², —NR²₂, -Halogen, —NO₂, —CN, —C(Halogen)₃, —C(O)R², —C(O)NHR², C(O)NR²₂, —NC(O)R², —S(O)₂NHR², —S(O)₂NR²₂, —S(O)₂R², —P(O)₂—R², —P(O)—R², —S(O)—R², P(O)—OR², —S(O)—OR², —N+R²₃, wherein R² is H, C₁-C₆ alkyl, C₂-C₆ alkenyl, C₂-C₆ alkynyl, or aryl,
C-F-connecting group is chosen from the group consisting of —SO₂—O—, —O—SO₂—O—, —C(O)—, —S+(R³)—, —C—U—C(V)—O—, —P⁺(W)₂—O—, —P(W)—O— where U is —C(R²)₂—, —NR²— or —O—; V is ═O or ═NR² and W is —OR² or —N(R²)₂
Functional entity precursor is —C(H)(R³)—R⁴ or functional entity precursor is heteroaryl or aryl optionally substituted with one or more substituents belonging to the group comprising R³ and R⁴.
Wherein R³ and R⁴ independently is H, alkyl, alkenyl, alkynyl, alkadienyl, cycloalkyl, cycloheteroalkyl, aryl or heteroaryl, optionally substituted with one or more substituents selected from the group consisting of SnR⁵R⁶R⁷, Sn(OR⁵)R⁶R⁷, Sn(OR⁵)(OR⁶)R⁷, BR⁵R⁶, B(OR⁵)R⁶, B(OR⁵)(OR⁶), halogen, CN, CNO, C(halogen)₃, OR⁵, OC(═O)R⁵, OC(═O)OR⁵, OC(═O)NR⁵R⁶, SR⁵, S(═O)R⁵, S(═O)₂R⁵, S(═O)₂NR⁵R⁶, NO₂, N₃, NR⁵R⁶, N⁺R⁵R⁶R⁷, NR⁺R⁶, NR⁵NR⁶R⁷, NR⁵C(═O)R⁶, NR⁵C(═O)OR⁶, NR⁵C(═O)NR⁶R⁷, NC, P(═O)(OR⁵)OR⁶, P+R⁵R⁶R⁷, C(═O)R⁵, C(═NR⁵)R⁶, C(═NOR⁵)R⁶, C(═NNR⁵R⁶), C(═O)OR⁵, C(═O)NR⁵R⁷, C(═O)NR⁵R⁶, C(═O)NR⁵NR⁶R⁷, C(═NR⁵)NR⁶R⁷, C(═NOR⁶)NR⁶R⁷ or R⁸,
wherein,
R⁵, R⁵, and R⁷ independently is H, alkyl, alkenyl, alkynyl, alkadienyl, cycloalkyl, cycloheteroalkyl, aryl or heteroaryl, optionally substituted with one or more substituents selected from the group consisting of halogen, CN, CNO, C(halogen)₃, ═O, OR⁸, OC(═O)R⁸, OC(═O)OR⁸, OC(═O)NR⁸R⁹, SR⁸, S(═O)R⁸, S(═O)₂R⁸, S(═O)₂NR⁸R⁹, NO₂, N₃, NR⁸R⁹, N⁺R⁸R⁹R¹⁰, NR⁵OR⁸, NR⁵NR⁸R⁷, NR⁸C(═O)R⁹, NR⁸C(═O)OR⁹, NR⁸C(═O)NR⁹R¹⁰, NC, P(═O)(OR⁸)OR⁹, P⁺R⁵R⁶R⁷, C(═O)R⁸, C(═NR⁸)R⁹, C(═NOR⁸)R⁹, C(═NNR³R⁹), C(═O)OR⁸, C(═O)NR⁸R⁹, C(═O)NR⁸OR⁹C(═NR⁵)NR⁶R⁷, C(═NOR⁵)NR⁶R⁷ or C(═O)NR⁸NR⁹R¹⁰, wherein R⁵ and R⁶ may together form a 3-8 membered heterocyclic ring or R⁵ and R⁷ may together form a 3-8 membered heterocyclic ring or R⁶ and R⁷ may together form a 3-8 membered heterocyclic ring,
wherein,
R⁸, R⁹, and R¹⁰ independently is H, alkyl, alkenyl, alkynyl, alkadienyl, cycloalkyl, cycloheteroalkyl, aryl or heteroaryl and wherein R³ and R⁹ may together form a 3-8 membered heterocyclic ring or R⁸ and R¹⁰ may together form a 38 membered heterocyclic ring or R⁹ and R^{10 may together form a} 3-8 membered heterocyclic ring.

2. A compound according to claim 1 wherein, Functional entity precursor is —C(H)(R¹¹)—R¹¹, or functional entity precursor is heteroaryl or aryl substituted with 0-3 R¹¹, 0-3 R¹³ and 0-3 R¹⁵, wherein
R¹¹ and R¹¹, are independently H, or selected among the group consisting of a C₁-C₆ alkyl, C₂-C₈ alkenyl, C₂-C₆ alkynyl, C₄-C₈ alkadienyl, C₃-C₇ cycloalkyl, C₃-C₇ cyclo-heteroalkyl, aryl, and heteroaryl, said group being substituted with 0-3 R¹², 0-3 R¹³ and 0-3 R¹⁵,
or R¹¹ and R¹¹, are C₁-C₃ alkylene-NR¹²₂, C₁-C₃ alkylene-NR¹²C(O)R¹⁶, C₁-C₃ alkylene-NR¹²C(O)OR¹⁶, C₁-C₂ alkylene-O—NR¹²₂, C₁-C₂ alkylene-O—NR¹²C(O)R¹⁶, C₁-C₂ alkylene-O—NR¹²C(O)OR^{1r} substituted with 0-3 R¹⁵,
where R¹² is H or selected independently among the group consisting of C₁-C₆ alkyl, C₂-C₈ alkenyl, C₂-C₆ alkynyl, C₃-C₇ cycloalkyl, C₃-C₇ cycloheteroalkyl, aryl, heteroaryl, said group being substituted with 0-3 R¹³ and 0-3 R¹⁵,
R¹³ is selected independently from —N₃, —CNO, —C(NOH)NH₂, —NHOH, —NHNHR¹⁷, —C(O)R¹⁷, —SnR¹⁷₃, —B(OR¹⁷)₂, —P(O)(OR¹⁷)₂ or the group consisting of C₂-C₆ alkenyl, C₂-C₆ alkynyl, C₄-C₈ alkadienyl said group being substituted with 0-2 R¹⁴
where R¹⁴ is independently selected from —NO₂, —C(O)OR¹⁷, —COR¹⁷, —CN, —OSiR¹⁷₃, —OR¹⁷ and —NR¹⁷₂;
R¹⁵ is ═O, —F, —Cl, —Br, —I, —CN, —NO₂, —OR¹⁷, —NR⁷₂, —NR¹⁷—C(O)R¹⁶, —NR¹⁷—C(O)OR¹⁶, —SR¹⁷, —S(O)R¹⁷, —S(O)₂R¹⁷, —COOR¹⁷, —C(O)NR¹⁷₂ and —S(O)₂NR¹⁷₂,
R¹⁶ is H, C₁-C₆ alkyl, C₂-C₆ alkenyl, C₂-C₈ alkynyl, C₃-C₇ cycloalkyl, aryl or C₁-C₆ alkylene-aryl substituted with 0-3 substituents independently selected from —F, —Cl, —NO₂, —R², —R², —SiR²₃;
R¹⁷ is selected independently from H, C₁-C₆ alkyl, C₃-C₇ cycloalkyl, aryl, C₁-C₆ alkylene-aryl, 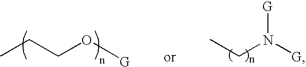
G is H or C₁-C₆ alkyl and n is 1, 2, 3 or 4.

3. A compound according to claim 2 wherein, Functional entity precursor is —C(H)(R¹¹)—R¹¹, or functional entity precursor is heteroaryl or aryl substituted with 0-3 R¹¹, 0-3 R¹³ and 0-3 R¹⁵, wherein
R¹¹ and R¹¹, are independently H, or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cyclo-heteroalkyl, aryl, and heteroaryl, said group being substituted with 0-3 R¹², 0-3 R¹³ and 0-3 R¹⁵,
or R¹¹ and R¹¹, are C₁-C₃ alkylene-NR¹²₂, C₁-C₃ alkylene-NR¹²C(O)R¹⁶, C₁-C₃ alkylene-NR¹²C(O)OR¹⁶, C₁-C₂ alkylene-O—NR¹²₂, C₁-C₂ alkylene-O—NR¹²C(O)R¹⁶, C₁-C₂ alkylene-O—NR¹²C(O)OR¹⁶ substituted with 0-3 R¹⁵,
where R¹² is H or selected independently among the group consisting of C₁-C₆ alkyl, C₂-C₈ alkenyl, C₂-C₆ alkynyl, C₃-C₇ cycloalkyl, C₃-C₇ cycloheteroalkyl, aryl, heteroaryl, said group being substituted with 0-3 R¹³ and 0-3 R¹⁵, —
R¹³ is selected independently from —N₃, —CNO, —C(NOH)NH₂, —NHOH, —NHNHR¹⁷, —C(O)R¹⁷, —SnR¹⁷₂, —P(OR¹⁷)₂, —P(O)(OR¹⁷)₂ or the group consisting of C₂-C₆ alkenyl, C₂-C₆ alkynyl, C₄-C₈ alkadienyl said group being substituted with 0-2 R¹⁴
where R¹⁴ is independently selected from —NO₂, —C(O)OR¹⁷, —COR¹⁷, —CN, —OSiR¹⁷₃₁—OR¹⁷ and —NR¹⁷₂;
R¹⁵ is ═O, —F, —Cl, —Br, —I, —CN, —NO₂, —OR¹⁷, —NR¹⁷₂, —NR¹⁷—C(O)R¹⁶, —NR¹⁷—C(O)OR¹⁶, —SR¹⁷, —S(O)R¹⁷, —S(O)₂R¹⁷, —COOR¹⁷, —C(O)NR¹⁷₂ and —S(O)₂NR¹⁷₂,
R¹⁶ is H, C₁-C₈ alkyl, C₂-C₆ alkenyl, C₂-C₆ alkynyl, C₃-C₇ cycloalkyl, aryl or C₁-C₆ alkylene-aryl substituted with 0-3 substituents independently selected from —F, —Cl, —NO₂, —R², —OR², —SiR²₃;
wherein R¹⁷ is selected independently from H, C₁-C₈ alkyl, C₃-C₇ cycloalkyl, aryl, C₁-C₆ alkylene-aryl.

4. A compound according to claim 1 wherein, Functional entity precursor is —C(H)(R¹¹)—R¹¹, wherein
R¹¹ and R¹¹, are or C₁-C₃ alkylene-NR¹²₂, C₁-C₃ alkylene-NR¹²C(O)R¹⁶, C₁-C₃ alkylene-NR¹²C(O)OR¹⁶, C₁-C₂ alkylene-O—NR¹²₂, C₁-C₂ alkylene-O—NR¹²C(O)R¹⁶, C₁-C₂ alkylene-O—NR¹²C(O)OR¹⁶ substituted with 0-3 R¹⁵

5. A compound according to claim 1 wherein, Functional entity precursor is —C(H)(R¹¹)—R¹¹, wherein
R¹¹ and R¹¹, are independently H, or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cyclo-heteroalkyl, aryl, and heteroaryl, said group being substituted with 0-3 R¹², 0-3 R¹³ and 0-3 R¹⁵,

6. A compound according to claim 2 wherein, Functional entity precursor is —C(H)(R¹¹)—R¹¹, wherein
R¹¹ and R¹¹, are independently H, or selected among the group consisting of a C₁-C₆ alkyl, C₃-C₇ cycloalkyl, C₃-C₇ cycloheteroalkyl, aryl, and heteroaryl, said group being substituted with 0-3 R¹² and 0-3 R¹⁵,
where R¹² is H or selected independently among the group consisting of C₁-C₆ alkyl, C₂-C₆ alkenyl, C₂-C₆ alkynyl, C₃-C₇ cycloalkyl, C₃-C₇ cycloheteroalkyl, aryl, heteroaryl.
R¹⁵ is ═O, —F, —Cl, —Br, —I, —CN, —NO₂, —OR¹⁷, —NR¹⁷₂, —NR¹⁷—C(O)R¹⁶, —NR¹⁷—C(O)OR¹⁶, —SR¹⁷, —S(O)R¹⁷, —S(O)₂R¹⁷, —C(O)R¹⁷, —C(O)NR¹⁷₂ and —S(O)₂NR¹⁷₂, R¹⁷ is selected independently from H, C₁-C₆ alkyl, C₃-C₇ cycloalkyl, C₁-C₆ alkylene-aryl,

7. A compound according to claim 1 wherein, Functional entity precursor is heteroaryl or aryl substituted with 0-3 R¹¹, 0-3 R¹³ and 0-3

8. A compound according to claim 2 wherein C-F-connecting group is chosen from the group consisting of —SO₂—O—, —O—SO₂—O—, —C(O)—O—, —S⁺(R¹¹)—, —C—U—C(V) —O—, —P⁺(W)₂—O—, and —P(W)—O— where U is —C(R²)₂—, —NR²— or —O—; V is ═O or ═NR² and W is —OR² or —N(R²)₂

9. A compound according to claim 2 wherein C-F-connecting group is —S⁺(R¹¹)—,

10. A compound according to claims 1-2 wherein C-F-connecting group is chosen from the group consisting of —SO₂—O—, —O—SO₇—O—, —C(O)—, —S+(R¹⁷)—, —C—U—C(V)—O—, —P⁺(W)₂—O—, and —P(V)—O— where U is —C(R²)₂—, —NR²— or —O—; V is ═O or ═NR² and W is —OR² or 4N(R²)₂, wherein R¹⁷ is selected independently from H, C₁-C₆ alkyl, C₃-C₇ cycloalkyl, aryl, C₁-C₆ alkylene-aryl.

11. A compound according to claims 1-2 wherein C-F-connecting group is chosen from the group consisting of —SO₂—O—, and —S+(R¹⁷)—; wherein R¹⁷ is selected independently from H, C₁-C₆ alkyl, C₃-C₇ cycloalkyl, aryl, C₁-C₆ alkylene-aryl.

12. A compound according to claim 1 wherein, Spacer is a valence bond, C₁-C₆ alkylene-A-, C₂-C₆ alkenylene-A-, C₂-C₈ alkynylene-A-, or 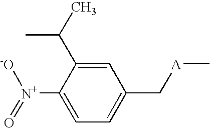
said spacer optionally being connected through A to a linker selected from —(CH₂)ₙ—B—, 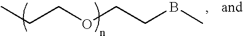
—(CH₂)ₙ—S—S—(CH₂)ₘ—B—
where A is a valence-bond, —C(O)NR¹⁷—, —NR¹⁷—, —O—, —S—, or —C(O)—O—; B is avalence bond, —O—, —S—, —NR¹⁷— or —C(O)NR¹⁷— and connects to S-C-connecting group; and n and m independently are integers ranging from 1 to 10; and R¹⁷ is selected independently from H, C₁-C₆ alkyl, C₃-C₇ cycloalkyl, aryl, or C₁-C₆ alkylene-aryl

13. A compound according to claim 1 wherein, Spacer is a valence bond, C₁-C₆ alkylene-A-, C₂-C₆ alkenylene-A-, C₂-C₆ alkynylene-A-, or 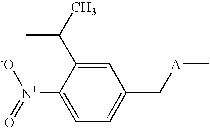
said spacer optionally being connected through A to a linker selected from —(CH₂)ₙ—B—, 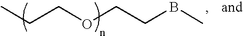
where A is a valence bond, —C(O)NR¹⁷—, —NR¹⁷—, —S—, or —C(O)—O—; B is −O—S—, —NR¹⁷—, or —C(O)NR¹⁷— and connects to S-C-connecting group; and n and m independently are integers ranging from 1 to 6; and R¹⁷ is selected independently from H, C₁-C₆ alkyl, C₃-C₇ cycloalkyl, aryl, or C₁-C₆ alkylene-aryl

14. A compound according to claim 1-2 wherein, S-C-connecting group is a valence bond, —NH—C(═O)—, —NH—SO₂—, —S—S—, 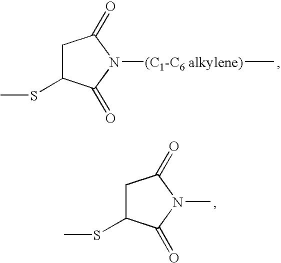
—C(═O)—NH—, or

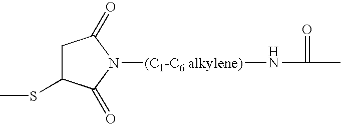

15. A compound according to claim 2 wherein, the carrier is selected from the group consisting of arylene, heteroarylene or —(CF₂)ₘ. substituted with 0-3 R¹ wherein m is an integer between 1 and 10, and C-F-connecting group is —O₂—O—, and the functional entity precursor is —C(H)(R¹¹)—R¹¹.

16. A compound according to claim 2 wherein, the carrier is —(CF₂)ₘ— wherein m is an integer between 1 and 10, the C-F-connecting group is —SO₂—O—; and the functional entity precursor is aryl or heteroaryl substituted with 0-3 R¹¹, 0-3 R¹³ and 0-3 R¹⁵.

17. A compound according to claims 1-16 wherein Complementing element is a nucleic acid.

18. A compound according to claims 1-16 where Complementing element is a sequence of nucleotides selected from the group of DNA, RNA, LNA PNA, or morpholino derivatives.

19. A library of compounds according to claim 1, wherein each different member of the library comprises a complementing element having a unique sequence of nucleotides, which identifies the functional entity.

20. A method for transferring a functional entity precursor to a recipient reactive group, comprising the steps of
providing one or more building blocks according to claims 1 to 18,
contacting the one or more building blocks with a corresponding encoding element associated with a recipient reactive group under conditions which allow for a recognition between the one or more complementing elements and the encoding elements, said contacting being performed prior to, simultaneously with, or subsequent to a transfer of the functional entity precursor to the recipient reactive group.

21. The method according to claim 20, wherein the encoding element comprises one or more encoding sequences comprised of 1 to 50 nucleotides and the one or more complementing elements comprises a sequence of nucleotides complementary to one or more of the encoding sequences.

22. The method of claims 20 or 21, wherein the recipient reactive group is a nucleophilic S— or N— atom, which may be part of a chemical scaffold, and the activating catalyst is contains palladium.
